(19) United States
(12) Patent Application Publication
(10) Pub. No.: US 2024/0130801 A1
(43) Pub. Date: Apr. 25, 2024

(54) ROBOTIC ASSISTED IMAGING

(71) Applicant: Worcester Polytechnic Institute, Worcester, MA (US)

(72) Inventors: Haichong Zhang, Shrewsbury, MA (US); Xihan Ma, Worcester, MA (US); Ashiqur Rahaman, Worcester, MA (US); Wen-Yi Kuo, Worcester, MA (US)

(21) Appl. No.: 18/381,510

(22) Filed: Oct. 18, 2023

Related U.S. Application Data
(60) Provisional application No. 63/416,989, filed on Oct. 18, 2022.

(51) Int. Cl. A61B 34/20; A61B 8/00; A61B 10/02; A61B 34/32; A61B 90/00

(52) U.S. Cl. CPC A61B 34/20; A61B 8/4218; A61B 8/4263; A61B 10/0233; A61B 34/32; A61B 90/361; A61B 2034/2055; A61B 2034/2063; A61B 2034/2065

(57) ABSTRACT

An imaging self-positioning system includes a robotic actuator for manipulating an imaging tool or medical probe and a sensory component for maintaining a normal orientation above patient a treatment site. The imaging tool, typically an US probe, is grasped by an end-effector or similar actuator, and a sensory component engaged with the imaging tool senses an orientation of the tool relative to the treatment surface, and the robotic actuator disposes the imaging tool for maintaining a normal or other predetermined angular alignment with the treatment surface. The treatment surface is a patient epidermal region adjacent an imaged region for identifying anatomical features and surgical targets. A medical probe such as a biopsy needle may accompany the end-effector for movement consistent with the probe, either manually or robotically advanced towards the surgical target.

### RELATED APPLICATIONS

This patent application claims the benefit under 35 U.S.C. § 119(e) of U.S. Provisional Patent App. No. 63/416,989 filed Oct. 18, 2022, entitled “ROBOTIC ASSISTED IMAGING,” incorporated herein by reference in entirety.

### STATEMENT OF FEDERALLY SPONSORED RESEARCH

This invention was made with government support under grant DP5 OD028162, awarded by the National Institute for Health. The government has certain rights in the invention.

### BACKGROUND

Medical imaging has vastly improved medical diagnosis and treatment fields by allowing doctors and medical technicians to visualize internal anatomical structures. Among the many imaging capabilities available, ultrasound mediums are favored for their benign signals and portability. Ultrasound (US) imaging has been widely adopted for abnormality monitoring, obstetrics, guiding interventional, and radiotherapy procedures. US is acknowledged for being cost-effective, real-time, and safe. Nonetheless, the US examination is a physically demanding procedure. Sonographers needs to press the US probe firmly onto the patient's body and fine-tune the probe's image view in an un-ergonomic way. More importantly, the examination outcomes are heavily operator-dependent. The information contained in the US images can be easily affected by factors such as scan locations on the body, the probe orientations at the scan location and the contact force at the scan location. Obtaining consistent examination outcomes requires highly skilled personnel with substantial experience.

### SUMMARY

An imaging self-positioning system includes a robotic actuator for manipulating an imaging tool or medical probe and a sensory component for maintaining a normal orientation adjacent patient a treatment site. The imaging tool, typically an US probe, is grasped by an end-effector or similar actuator, and a sensory component engaged with the imaging tool senses an orientation of the tool relative to the treatment surface, and the robotic actuator disposes the imaging tool for maintaining a normal or other predetermined angular alignment with the treatment surface. The treatment surface is a patient epidermal region adjacent an imaged region for identifying anatomical features and surgical targets. A medical probe such as a biopsy needle may accompany the end-effector for movement consistent with the probe, either manually or robotically advanced towards the surgical target.

Robotic members are often sought for performing repetitive object placement tasks such as assembly and sorting of various objects or parts. Robot-assisted imaging may include a procedure using an end-effector of a robot arm or mechanical actuators to manipulate an imaging probe (for ultrasound, optics, and photoacoustic imaging) to realize teleoperative or autonomous tasks. Such a procedure employs sensing of the surface terrain (e.g., skin) and controlling both the orientation and location of the probe by grasping the probe through the end-effector, typically a claw or similar actuator.

Configurations herein are based, in part, on the observation that convention medical imaging, and in particular US imaging, is often employed by skilled sonographers for obtaining visual imaging for diagnosis and real time feedback during minimally invasive procedures using a needle or probe. Unfortunately, conventional approaches to US imaging suffer from the shortcoming that it can be problematic to manipulate an imaging probe for an accurate depiction of a surgical target, particularly during concurrent insertion of the needle or instrument. US probes, while portable, are dependent on accurate positioning at the treatment surface for rendering positional guidance. Accordingly, configurations herein substantially overcome the shortcoming of conventional US procedures by providing a self-positioning robotic apparatus for positioning and maintaining an alignment of the probe at a predetermined angle with the treatment site. Typically a normal or substantially normal orientation to the surface is sought, however an angular tilt may be beneficial to avoid anatomical structures obscuring the surgical target.

In a particular use case of a needle or instrument, insertion force is another parameter that eludes automation. Insertion progression and depth may be measured by resistance, or the force needed for insertion. However, varied densities of anatomical tissue, as well as variances due to an insertion angle, can make depth sensing based on resistive force to insertion unreliable.

In an example configuration, the imaging device performs a method for robotic positioning of a by receiving, from each plurality of sensing elements disposed in proximity to a medical instrument, a signal indicative of a distance to a treatment site of a patient. The controller computes, based on each of the signals and an offset of the sensor from the medical instrument, a distance from each of the respective sensing elements to the treatment site. The medical instrument may be an imaging probe, such that the imaging device determines, based on the computed distances, an angle of the medical instrument relative to the treatment site for optimal imaging alignment of a surgical site,

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of the self-orienting sensor device;

FIGS. 2A-2C are schematic diagrams of the imaging probe and end effector in the device of FIG. 1;

FIGS. 3A-3B are respective plan and side views of the integrated probe and position sensor ring of FIGS. 2A-2C;

FIGS. 4A-4D show sensor calibration for the position sensor ring of FIGS. 3A-3B;

FIGS. 5A-5B show an alternative sensor configuration employing video image sensors; and

FIGS. 6A and 6B depict comparisons of hand/manual scan and automated images.

### DETAILED DESCRIPTION

Conventional manual ultrasound (US) imaging is a physically demanding requiring skilled operators for accurate positioning of the imaging sensor. A Robotic Ultrasound system (RUSS) has the potential to overcome this limitation by automating and standardizing the imaging procedure. It also extends ultrasound accessibility in resource-limited environments with the shortage of human operators by enabling remote diagnosis. During imaging, maintaining the US probe in a normal orientation to the skin surface largely benefits the US image quality. However, an autonomous, real-time, low-cost method to align the probe towards the direction orthogonal to the skin treatment without pre-operative information is absent in conventional RUSS.

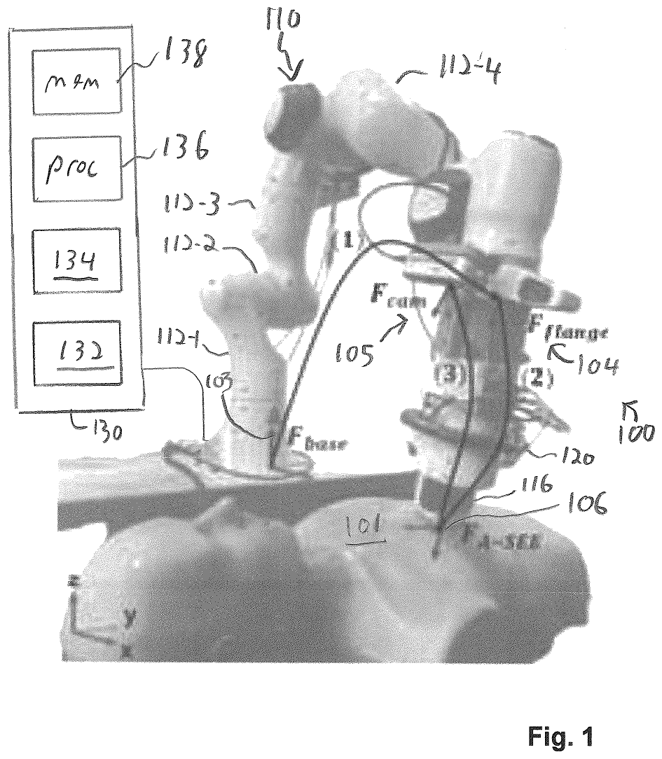

FIG. 1 is a context diagram of the self-orienting sensor device 100. The device 100 performs a method for robotic assisted medical imaging and procedures, including engaging an imaging probe with a robotic actuator such as an end-effector grasping the probe or instrument, and moving the robotic actuator to dispose the imaging sensor at a predetermined location relative to a patient imaging location. The actuator maintains the imaging probe at the predetermined relative location even during movement of the patient so that a trajectory or scan direction remains consistent.

Referring to FIG. 1, a robotic arm 110 has a series of jointed segments 112-1 . . . 112-4 for movement of an end-effector or actuator 114 engaging an imaging probe 116 (probe) 101 in proximity over a treatment surface 101. A sensory ring 120 defines a frame positioned to encircle the probe 116 and has a plurality of sensors for detecting a distance to the treatment surface. The sensory ring 120 forms a circular frame for disposing the sensors at a known radius from a longitudinal axis of the probe 116.

A controller 130 includes a robotic positioning circuit 132 and logic and an image processor 134, along with a processor 136 and memory 138 for containing instructions as described further below. The method for robotic positioning of a surgical instrument or probe 116 includes receiving, from each plurality of sensing elements disposed in proximity to the probe 116, a signal indicative of a distance to a treatment site 101 of a patient, and computing, based on each of the signals and an offset of the sensor from the medical instrument, a distance from each of the respective sensing elements to the treatment site. This determines a normal or off-normal position of the sensor ring, and hence the probe, with the treatment surface. Based on the computed distances, the processor 136 computes an angle of the probe 116 relative to the treatment site 101.

An autonomous RUSS has been explored to address the issues with the conventional US. RUSS utilizes robot arms to manipulate the US probe. The sonographers are thereby relieved of the physical burdens. The diagnosis can be done remotely, eliminating the need for direct contact with patients. The desired probe pose (position and orientation) and the applied force can be parameterized and executed by the robot arm with high motion precision. As a result, the examination accuracy and repeatability can be secured. The probe pose can also be precisely localized, which enables 3D reconstruction of human anatomy with 2D US images.

An autonomous scan may adopt a 2-step- strategy: First, a scan trajectory formed by a series of probe poses is defined using preoperative data such as Magnetic Resonance Imaging (MRI) of the patient or a vision-based point cloud of the patient body. Second, the robot travels along the trajectory while the probe pose and applied force are continuously updated according to intraoperative inputs (e.g., force/torque sensing, real-time US images, etc.). Yet, owing to factors including involuntary patient movements during scanning, inevitable errors in scan trajectory to patient registration, and a highly-deformable skin surface, which can be difficult to be measured preoperatively. The second step is of significance to the successful acquisition of diagnostically meaningful US images. The ability to update probe positioning and orientation in real-time is preferred to enhance the efficiency and safety of the scanning process. In particular, keeping the probe to an appropriate orientation assures a good acoustic coupling between the transducer and the body. A properly oriented probe position offers a clearer visualization of pathological clues in the US images. Real-time probe orientation adjustment is challenging and remains an open problem.

Configurations herein apply two aspects: i) a compact and cost-effective active-sensing end-effector (A-SEE) device that provides real-time information on the rotation adjustment required for achieving normal positioning. Conventional approaches do not achieve simultaneous in-plane and out-of-plane probe orientation control without relying on a passive contact mechanism; ii) the A-SEE approach integrates with the RUSS for implementing a complete US imaging workflow to demonstrate the A-SEE enabled probe self-normal-positioning capability. It should be further emphasized that normal positioning, meaning probe orientation locates a longitudinal axis of the probe at a normal, or perpendicular to a plane defined by the skin surface, is an example of a preferred orientation; other angular orientations may be determined.

FIG. 1 defines corresponding coordinate frames of reference. Coordinate frame F_{base} 103 corresponds to the robot base frame; F_{flange} 104 is the flange frame to attach the end-effector; F_{cam} 105 is an RGB-D camera's frame adjacent the end effector and F_{A-SEE} 106 is the US probe tip frame. The probe 116 orientation as controlled by the robot incorporates these frames as follows.

(1) depicts a transformation from F_{base} to F_{flange}, denoted as T^{base-flange}

(2) denotes a transformation from F_{flange} to F_{A-SEE}, denoted as T^{flange-A-see}

(3) is the transformation from F_{A-SEE} to F_{cam}, denoted as T^{A-see-cam}.

Operation of the controller includes the implementation details of A-SEE and its integration with a RUSS to manipulate the actuator 114 according to the sensor ring 120. A typical use case involves preoperative probe landing pose identification and intraoperative probe self-normal-positioning with contact force adaptation. During imaging, the shared control scheme can allow teleoperative sliding of the probe along the patient body surface, as well as rotating the probe about its axis. Of course, the normal (or other angle pose) can assist in in-person procedures as well.

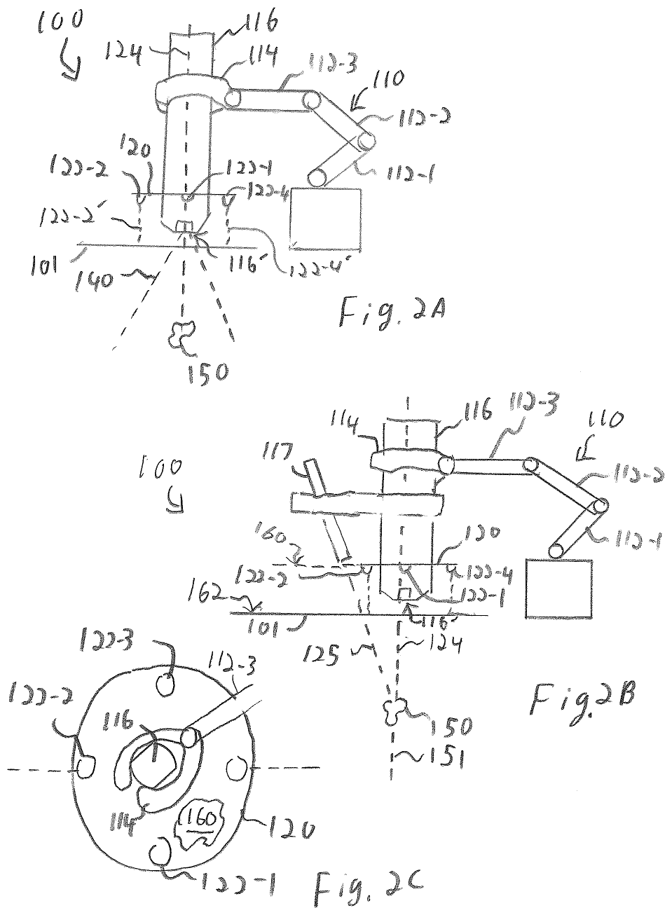

FIGS. 2A-2C are schematic diagrams of the imaging probe and end effector in the device of FIG. 1. Referring to FIGS. 1 and 2A, a plurality of sensing elements 122-1 . . . 122-4 (122 generally) are disposed in proximity to a medical instrument such as the probe 116. The sensory ring 120 positions the sensing elements in a predetermined orientation with a robotic actuator 114 when the robotic actuator engages the medical instrument. The actuator 114 engages or grabs the probe 116, and the sensory ring 120 attaches either to the probe 116 or the actuator 114 to define a predetermined orientation between the probe and sensors; in other words, the sensors 122 move with the probe 116 so that accurate positioning can be determined from the sensors. A particular configuration embeds four laser distance sensors 122 on the sensory ring 120 to estimate the desired positioning towards the normal direction, where the actuator is integrated with the RUSS system which allows the probe to be automatically and dynamically kept to a normal direction during US imaging. The actuator 114, and hence the probe 116, them occupies a known location relative to the sensors 122-1 . . . 122-4 (122 generally). Each of the sensors 122 then determines a signal indicative of a distance to the treatment site 101 of a patient.

A typical scenario deploys the probe 116 to have an imaging field 140 capturing images of a surgical target 150, usually a mass or anatomical region to be biopsied or pierced, although any suitable anatomical location may be sought. This usually involves identifying an axis 124 of the medical instrument or probe 116, such that the axis 124 extends towards the treatment site 101, and is based on an orientation of the axis 124 relative to the plane of the treatment site 101. The probe axis 124 is defined by a longitudinal axis through the center of mass of the probe 116, or other axis that denotes a middle of the sensed imaging field 140. In a simplest case, seeking a normal orientation of the probe 116 to the surface 101, each of the 4 distance sensors 122 returns an equal value. Differing values can give an angular orientation of the probe axis 124 relative to the treatment surface 101, as the “tilt” or angle of the sensory ring 120 will be reflected in the relative distance 122′-1 . . . 122′-4 (122 generally).

Either a sensory probe such as the US probe 116, or a surgical medical instrument such as a needle may be grasped by the actuator 114. The probe axis 124 therefore defines an approach angle of the medical instrument to the treatment site 101, where the sensors 122 are used to dispose the medical instrument based on a target angle defined by intersection of the axis 124 with the treatment site 101. The robotic arm 110 translates the surgical instrument along the axis 124, and therefore disposes the robotic actuator 114 based on the determined angle of the medical instrument.

FIG. 2B shows a probe 116 in conjunction with a needle 117 or other medical or surgical instrument, or elongated shaft. When the needle 117 is attached via a bracket 118 or similar fixed support, the probe 116 and needle 117 share the same frame of reference for relative movement. Referring to FIGS. 1-2B, such a procedure may include identifying the surgical target 150, where the surgical target 150 is disposed on an opposed side of the plane defining the treatment surface 101, meaning beneath the patients skin, The probe axis 124 aligns with an axis 151 leading to the surgical target, disposing the medical instrument 117 for aligning an axis 125 with the treatment site 101, and advancing the medical instrument along the axis aligned with the treatment site and intersecting with the probe axis 124 at the surgical target 140.

The probe axis 124 need not be normal to the treatment surface 101. In general, the probe 116 receives a location of the surgical target 150 in the imaging region 140. The sensors 122 may be used to compute the angle of the medical instrument based on an intersection with the surgical target 150 and the probe axis 124. The medical instrument 117 may then be projected along the computed angle for attaining the surgical target 150.

In FIG. 2C, an example of the sensory ring 120 is shown. While three points define a plane, the use of 4 sensors allows a pair of sensors to align with a sensory plane of the imaging region 140, and the unaligned pair of sensors (offset)90° then provides an angular position of the imaging plane. Additional sensors could, of course, be employed. A probe plane is defined by the plurality of sensors 122 and the sensory ring 120. The sensory ring 120 encircles the probe 116 and at a known distance from an imaging tip 116′ or US sensor. Once the actuator 114 grasps or engages the probe 116, and the sensory ring 120 is secured around the probe, the controller 130 can determine an orientation of the medical instrument to the probe plane (sensor location). It then identifies a patient plane defined by the treatment site based on the sensor 122 distances. This allows computing an orientation of a probe plane 160 relative to the patient plane 162 based on the computed distances. 122′.

Any suitable sensing medium may be employed for the sensors 122. In an example configuration, optical based sensors such as infrared (IR) are a feasible option, however other mediums such as laser, electromagnetic or capacitance can suffice given appropriate power and distance considerations.

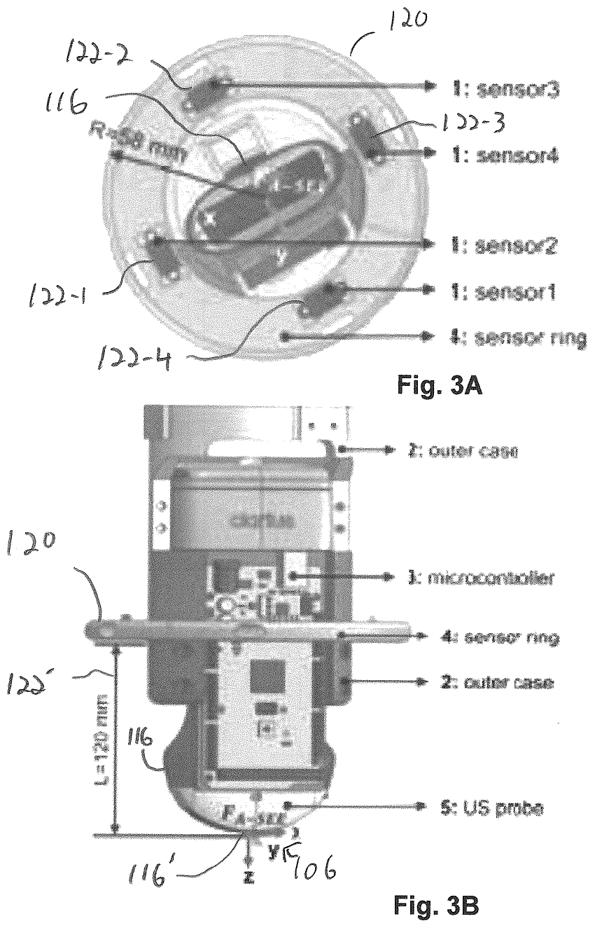

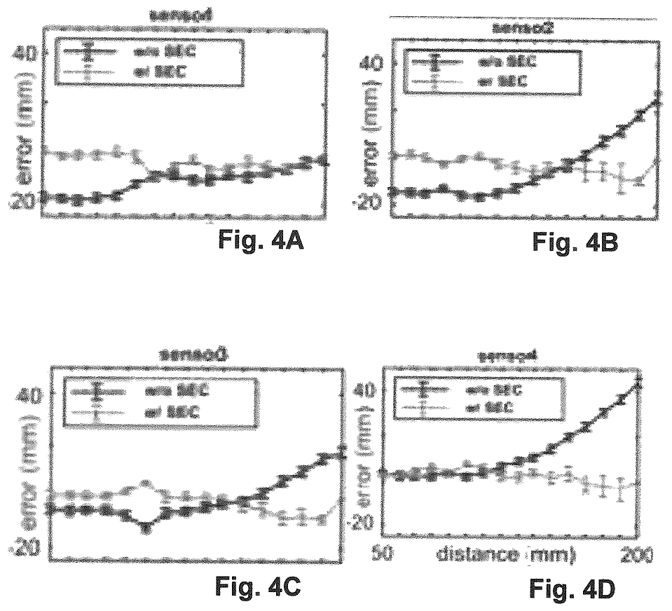

FIGS. 3A-3B are respective plan and side views of the integrated probe and position sensor ring of FIGS. 2A-2B integrated in an imaging device 100 as in FIG. 1. Referring to FIGS. 1-3B, the device 100 engages the medical instrument (probe) 116 with a robotic actuator 114 for advancing the medial instrument. Since the probe orientation is adjusted based on the sensor readings, the normal positioning performance depends largely on the distance sensing accuracy of the sensors. The purpose of sensor calibration is to model and compensate for the distance sensing error so that the accuracy can be enhanced. First, a trial is conducted to test the accuracy of each sensor, where a planar object was placed at different distances (from 50 mm to 200 mm with 10 mm intervals measured by a ruler). The sensing errors were calculated by subtracting the sensor readings from the actual distance. The 50 to 200 mm calibration range is experimentally determined to allow 0 to 60 degrees arbitrary tilting of A-SEE on a flat surface without letting the sensor distance readings exceed this range. Distance sensing beyond this range will be rejected. The results of the sensor accuracy test are shown in FIGS. 4A-4D. Referring to FIGS. 4A-4D, black curves indicate that the sensing error changes at different sensing distances with a distinctive distance-to-error mapping for each sensor. A sensor error compensator (SEC) is designed in the form of a look-up table that stores the sensing error versus the sensed distance data. SEC linearly interpolates the sensing error given arbitrary sensor distance input. The process of reading the look-up table is described by f:d_>∈R⁴→e_>∈R⁴, where d_> stores the raw sensor readings; e_> stores the sensing errors to be compensated. The sensor reading with SEC applied is given by:

$$\vec{d} = \{ \begin{matrix}\overline{d} + f ( \overline{d} ) & {d}_{min} <= \overline{{d}_{0}} <= {d}_{max} \\ {d}_{min} & \overline{{d}_{0}} < {d}_{min} \\ {d}_{max} & \overline{{d}_{0}} > {d}_{max}\end{matrix}$$

where dₘᵢₙ is 50 mm, dₘax is 200 mm. With SEC, the same trials were repeated. The curves in FIGS. 4A-4D show the sensing accuracy. The mean sensing error was 11.03±1.61 mm before adding SEC and 3.19±1.97 mm after adding SEC. A two-tailed t-test (95% confidence level) hypothesizing no significant difference in the sensing accuracy with and without SEC was performed. A p-value of 9.72×10⁻⁸ suggests SEC can considerably improve the sensing accuracy.

The values in FIGS. 4A-4D show curves for the respective sensors 122-1 . . . 122-4 (sensors 1-4) for distance measurement error before and after adding sensor error compensator. Having accurate distance readings from the sensors in real-time, A-SEE can be integrated with the robot to enable “spontaneous” motion that tilts the US probe towards the normal direction of the skin surface. A moving average filter is applied to the estimated distances to ensure motion smoothness. As depicted in FIGS. 2A-2C, upon normal positioning of the probe 116, the distance differences between sensor1, and 3, sensor2, and 4 are supposed to be minimized. This is facilitated by simultaneously applying in-plane rotation, which generates angular velocity about the y-axis of F_{A-SEE} (ω_{ny}), and out- of-plane rotation, which generates angular velocity about the x-axis of F_{A-SEE} (ωₙₓ). The angular velocities about the two axes at timestamp t are given by a PD control law:

$$[ \begin{matrix}{ω}_{nx} \\ {ω}_{ny}\end{matrix} ] = [ \begin{matrix}{K}_{p} & {K}_{d} & 0 & 0 \\ 0 & 0 & {K}_{p} & {K}_{d}\end{matrix} ] [ \begin{matrix}{d}_{13} ( t ) \\ \frac{Δ {d}_{13} ( t )}{Δ t} \\ {d}_{24} ( t ) \\ \frac{Δ {d}_{24}}{Δ t}\end{matrix} ]$$

where Kₚ and K_{d} are empirically tuned control gains;

d₁₃(t)=

[symbol]

d₃(t)−d₁(t), d₂₄(t)=

d₄(t)−d₂(t); Δd₁₃=

d₁₃(t)−d₁₃(t−1), Δd₂₄=

d₂₄(t)−d₂₄(t−1);

where:
d₁ to d₄ are the filtered distances from sensor 1 to 4, respectively; Δt is the control interval. ωₙₓ and ω_{ny} are limited within 0.1 rad/s. The angular velocity adjustment rate can reach to 30 Hz.

To prevent a loose contact between the probe and the skin that may cause acoustic shadows in the image, a force control strategy is necessary to stabilize the probe by pressing force at an adequate level throughout the imaging process. This control strategy is also responsible for landing the probe gently on the body for the patient's safety. A force control strategy is formulated to adapt the linear velocity along the z-axis expressed in F_{A-SEE}. The velocity adaptation is described by a two-stage process that manages the landing and the scanning motion separately: during landing, the probe velocity will decrease asymptotically as it gets closer to the body surface; during scanning, the probe velocity is altered based on the deviation of the measured force from the desired value.

Therefore, the velocity at time stamp t is calculated as:

ν_{fz}(t)=w·ν+(1−w)·ν_{fz}(t−1)

where w is a constant between 0 to 1 to maintain the smoothness of the velocity profile; ν is computed by:

$$v = \{ \begin{matrix}{K}_{p 1} ( \tilde{d} - min ( \overline{{d}^{′}} ) & min ( \overline{{d}^{′}} ) >= \overline{d} \\ {K}_{p 2} ( \tilde{F} - \overline{{F}_{2}} ) & min ( \overline{{d}^{′}} ) < \overline{d}\end{matrix}$$

Where d_′ is the vector of the four sensor readings after error compensation and filtering, and F⁻z is the robot measured force along the z-axis of F_{A-SEE}, internally estimated from joint torque readings. It is then processed using a moving average filter; F^{˜} is the desired contact force; Kₚ₁, Kₚ₂ are the empirically given gains; d^{˜} is the single threshold to differentiate the landing stage from the scanning stage, which is set to be the length from the bottom of the sensor ring to the tip 116′ of the probe (120 mm, in the example use case of FIG. 3B).

The combination of the self-normal-positioning and contact force control of the probe forms an autonomous pipeline that controls 3-DoF probe motion. A shared control scheme is implemented to give manual control of the translation along the x-, y-axis, and the rotation about the z-axis in concurrence with the three automated DoFs. A 3-DoF joystick may be used as an input source, whose movements in the three axes are mapped to the probe's linear velocity along the x-, y-axis (νₜₓ, ν_{ty}), and angular velocity about the z-axis (ω_{tz}), expressed in F_{A-SEE}.

A configuration of the imaging device 100 of FIG. 1 for providing 6-DoF control of the US probe is built by incorporating self-normal-positioning, contact force control, and teleoperation of the probe 116. In a use case, for a preoperative step, the patient lies on the bed next to the robot with the robot at its home configuration, allowing the RGB-D camera to capture the patient body. The operator selects a region of interest in a camera view as an initial probe landing position. By leveraging the camera's depth information, the landing position in 2D image space is converted to T^{cam} representing the 3D landing pose above the patient body relative to F_{cam}. The landing pose relative to F_{base} is then obtained by:

T_{land}^{base}=T_{flange}^{base}T_{A-SEE}^{flange}T_{cam}^{A-SEE}T_{land}^{cam}

Where T_{A-SEE}^{flange} and T_{cam}^{A-SEE} and are calibrated from a CAD model or measurements of the device 100. The robot then moves the probe 100 to a landing pose using a velocity-based PD controller. In the intraoperative step, the probe will be gradually attached to the skin using the landing stage force control strategy. Once the probe is in contact with the body, the operator can slide the probe on the body and rotate the probe about its long axis via the joystick. Meanwhile, commanding robot joint velocities generates probe velocities in F_{A-SEE}, such that the probe will be dynamically held in the normal direction and pressed with constant force. The desired probe velocities are formed as:

$$[ \begin{matrix}{v}^{A - \overline{S} EE} \\ {ω}^{A - \vec{S} EE}\end{matrix} ] = {[ {v}_{tx} {v}_{ty} {v}_{fz} {v}_{tx} {ω}_{nx} {ω}_{ny} {ω}_{tz} ]}^{T} .$$

Transforming them to velocities expressed in F_{base} yields:

$$[ \begin{matrix}{v}^{b \vec{a} se} \\ {ω}^{b \vec{a} se}\end{matrix} ] = [ \begin{matrix}{R}_{A - SEE}^{base} \cdot ( {v}^{A - \overline{S} EE} + ( {ω}^{A - \vec{S} EE} \times \overline{r} ) ) \\ {R}_{A - SEE}^{base} \cdot {ω}^{A - \vec{S} EE}\end{matrix} ]$$

Where R_{A-SEE}^{base} ∈ SO(3) is the rotational component of T_{A-SEE}^{base} ∈ SE(3); r̅ is given by:

[ [symbol] 1]^{T}=[0 0 0 1]^{T}

Lastly, the joint-space velocity command _q^{⋅} that will be sent to the robot for execution is obtained by:

$$\dot{\vec{q}} {J ( \vec{q} )}^{†} [ \begin{matrix}{v}^{b \vec{a} se} \\ {ω}^{b \vec{a} se}\end{matrix} ]$$

where J(_q)^{†} is the Moore-Penrose pseudo-inverse of the robot Jacobian matrix. During the scanning, the US images are streamed and displayed to the operator. The operator decides when to terminate the procedure. The robot will move back to its home configuration after completing the scanning.

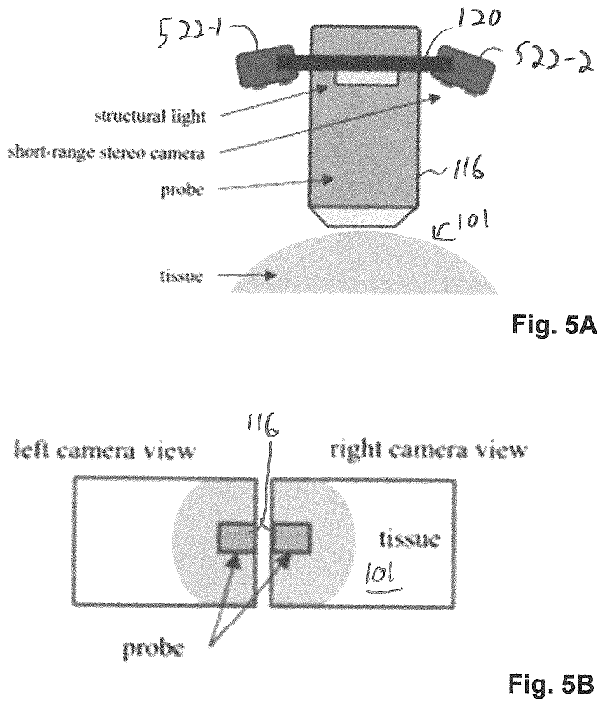

FIGS. 5A-5B show an alternative sensor configuration employing video image sensors. When US imaging is robotically enabled, as in configurations above, tagged as A-SEE. A remote operation is enabled. When integrated with a robotic manipulator, the A-SEE enables simplified operation for telesonography tasks: the sonographer operator only needs to provide translational motion commands to the probe, whereas the probe's rotational motion is automatically generated using A-SEE. This largely reduces the spatial cognitive burden for the operators and allows them to focus on the image acquisition task.

The example A-SEE device 100 employs single-point distance sensors to provide sparse sensing of the local contact surface. Such sparse sensing is sufficient to enable probe rotational autonomy when scanning flat, less deformable surfaces. However, dense sensing capability is needed when dealing with more complicated scan surfaces. To this end, the sparsely configured single-point distance sensors can be replaced with short-range stereo cameras (e.g., RealSense D405, Intel, USA), allowing dense RGB-D data acquisition of the probe's surroundings. In general, the plurality of sensing elements 122 define a set of points, such that each point of the set of points has a position and corresponding distance 122′ to the treatment site 101. In the configuration of FIGS. 5A and 5B, the distance 122′ signal is a video signal and the set of points defines a pixelated grid, such that the pixelated grid has a two dimensional representation of the position of a respective point in the set of points, i.e. similar to the 4 points of the sensors 122-1 . . . 122-4 with greater granularity. A non-tissue background can be precisely filtered out according to the RGB data, providing more accurate probe orientation control. The dense depth information can be used for the reconstruction of complex surfaces, facilitating the imaging of highly curved surfaces such as neck and limbs. In addition, the temporal aggregation of the depth information makes it possible to continuously track tissue deformation, allowing the imaging of highly deformable surfaces like the abdomen. Moreover, tracked deformation can be utilized to determine the appropriate amount of pressure to be applied on the body to receive optimal image quality without causing pain to the patient.

A conceptual graph of the dense-sensing A-SEE is shown in FIG. 5. Two short-range stereo cameras 522-1 . . . 522-2 are attached to the two sides of the probe 116. Merging the left and right camera views allows for the creation of a comprehensive representation of the probe region on the treatment site 101, including a panoramic color image and a panoramic depth map. Additionally, a light source is mounted in between the cameras to ensure adequate lighting, hence accurate depth map generation. The stereo camera based setup is approximately of the same dimension compared to the single-point distance sensor solution, and can be easily integrated with the robot.

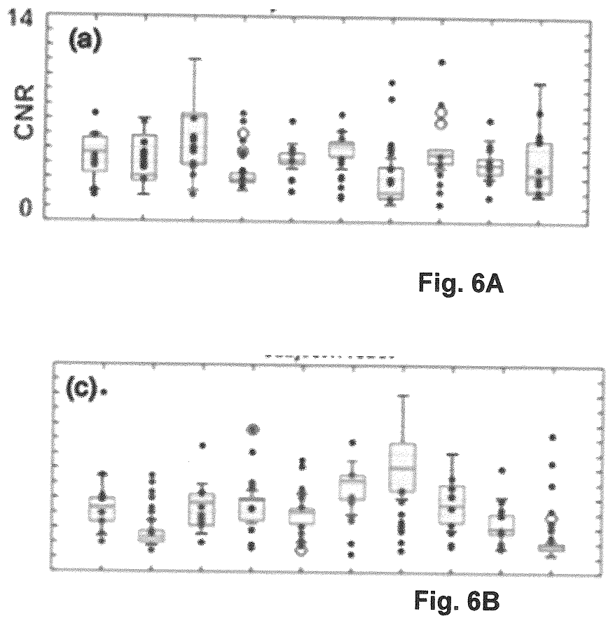

FIGS. 6A and 6B depict comparisons of hand/manual scan and automated images, respectively, captured as in FIGS. 1-4D. To assess the diagnostic quality of the acquired images, the contrast-noise-ratio (CNR) is employed to measure the image quality of the A-SEE tele-sonography system and is then compared to the images obtained through freehand scanning. FIGS. 6A and 6B show that lung images acquired with the A-SEE tele-sonography system (CNR: 4.86±2.03) (FIG. 6B) are not significantly different compared with images obtained by freehand scans (CNR: 5.20±2.58) of FIG. 6A.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for robotic positioning of a medical probe or instrument, comprising:
receiving, from each of a plurality of sensing elements disposed in proximity to a medical instrument, a signal indicative of a distance to a treatment site of a patient;
computing, based on each of the signals and an offset of the sensor from the medical instrument, a distance from each of the respective sensing elements to the treatment site; and
determining, based on the computed distances, an angle of the medical instrument relative to the treatment site.

2. The method of claim 1 further comprising identifying an axis of the medical instrument, the axis extending towards the treatment site, the angle based on an orientation of the axis relative to a plane defined by the treatment site.

3. The method of claim 2 wherein the axis defines an approach angle of the medical instrument, further comprising:
disposing the medical instrument at the angle based on a target angle defined by intersection of the axis with the treatment site; and
translating the surgical instrument along the axis.

4. The method of claim 2 further comprising:
identifying a surgical target, the surgical target disposed on an opposed side of the plane defining the treatment surface; and
disposing the medical instrument for aligning the axis with the treatment site; and
advancing the medical instrument along the axis aligned with the treatment site.

5. The method of claim 1 further comprising:
identifying a probe plane defined by the plurality of sensors;
determining an orientation of the medical instrument to the probe plane
identifying a patient plane defined by the treatment site;
computing an orientation of the probe plane relative to the patient plane based on the computed distances.

6. The method of claim 1 further comprising:
positioning the sensing elements in a predetermined orientation with a robotic actuator;
engaging the medical instrument with the robotic actuator; and
disposing the robotic actuator based on the determined angle of the medical instrument.

7. The method of claim 1 further comprising:
receiving a location of a surgical target;
computing the angle of the medical instrument based on an intersection with the surgical target; and
advancing the medical instrument along the computed angle for attaining the surgical target.

8. The method of claim 7 further comprising:
engaging the medical instrument with a robotic actuator for advancing the medial instrument.

9. The method of claim 1 wherein the distance sensor is configured for at least one of optical, ultrasonic, or visual sensing.

10. The method of claim 1 further comprising receiving, from the plurality of sensing elements, a set of points, each point of the set of points having a position and corresponding distance to the treatment site.

11. The method of claim 10 wherein the signal is a video signal and the set of points defines a pixelated grid, the pixelated grid having a two dimensional representation of the position of a respective point in the set of points.

12. The method of claim 1 wherein plurality of sensing elements are arranged in a plane, the offset indicative of a relative position from the medical treatment.

13. The method of claim 1 wherein the medical instrument has an axis passing through a longitudinal dimension of the medical instrument , the axis extending towards the treatment site, the angle based on an orientation of the axis relative to a plane defined by the treatment site.

14. An imaging device, comprising:
a robotic end-effector response to a controller;
a sensory frame adapted for encircling an imaging probe having a longitudinal axis;
a plurality of distance sensors arranged on the sensory frame;
positioning logic in the controller for manipulating the longitudinal axis at a predetermined angle responsive to the set of sensors based on a sensed distance to a treatment site.

15. The device of claim 14 further comprising an imaging probe disposed in a fixed plane of reference with the sensory frame.

16. The device of claim 14 further comprising a surgical instrument aligned with the circular frame, the surgical instrument adapted for forward translation to a surgical target based on the predetermined angle.

17. The device of claim 14 wherein the sensors are optical sensors adapted to receive a signal indicative of a distance to the treatment site, the positioning logic adapted to compute a correspondence to the predetermined angle based on the respective signals and an offset radius of the sensors from the longitudinal axis.

18. The device of claim 14 wherein the imaging probe radiates an imaging field onto the treatment site, the imaging field defining a plane, the plane aligned with a pair of sensors on the circular frame.

19. The device of claim 14 further comprising aligning a plane defined by the circular frame at a parallel orientation to a plane defining the treatment surface.
